(19) United States
(12) Patent Application Publication
(10) Pub. No.: US 2011/0171274 A1
(43) Pub. Date: Jul. 14, 2011

(54) Fesoterodine Substantially Free of Dehydroxy Impurity

(75) Inventors: Praveen Kumar Neela, Andhra Pradesh (IN); Kishore Charugundla, Andhra Pradesh (IN); Udhaya Kumar, Pondicherry (IN); Nitin Sharadchandra Pradhan, Maharashtra (IN)

(73) Assignee: ACTAVIS GROUP PTC EHF, Hafnarfjorour (IS)

(21) Appl. No.: 13/055,075

(22) PCT Filed: Jul. 21, 2009
(86) PCT No.: PCT/IB2009/006546

§ 371 Date: Apr. 4, 2011

(30) Foreign Application Priority Data
Jul. 21, 2008 (IN) 1742/CHE/2008

(51) Int. Cl. A61P 43/00; C07C 69/28; C07C 67/52; C07C 67/14; A61K 31/222; A61K 9/00

(52) U.S. Cl. 424/400; 560/140; 514/546; 428/402

(57) ABSTRACT

Provided herein is an impurity of fesoterodine, fesoterodine dehydroxy impurity, 2-[(1R)-3-[bis(1-methylethy)amino]-1-phenylpropyl]-4-methylphenyl isobutyrate, and a process for preparing and isolating thereof. Provided further herein is a highly pure fesoterodine or a pharmaceutically acceptable salt thereof substantially free of fesoterodine dehydroxy impurity, process for the preparation thereof, and pharmaceutical compositions comprising highly pure fesoterodine or a pharmaceutically acceptable salt thereof substantially free of dehydroxy impurity. Provided also herein is a pharmaceutical composition comprising solid particles of pure fesoterodine fumarate substantially free of dehydroxy impurity, wherein 90 volume-percent of the particles (D90) have a size of less than about 200 microns.

### CROSS REFERENCE TO RELATED APPLICATION

This application claims the benefit of priority to Indian provisional application No. 1742/CHE/2008, filed on Jul. 21, 2008, which is incorporated herein by reference in its entirety.

### FIELD OF THE DISCLOSURE

Disclosed herein is an impurity of fesoterodine, fesoterodine dehydroxy impurity, 2-[(1R)-3-[bis(1-methylethyl)amino]-1-phenylpropyl]-4-methylphenyl isobutyrate, and a process for preparing and isolating thereof. Disclosed further herein is a highly pure fesoterodine or a pharmaceutically acceptable salt thereof substantially free of fesoterodine dehydroxy impurity, process for the preparation thereof, and pharmaceutical compositions comprising highly pure fesoterodine or a pharmaceutically acceptable salt thereof substantially free of dehydroxy impurity.

### BACKGROUND

U.S. Pat. No. 6,713,464 B1 discloses a variety of 3,3-diphenylpropylamine derivatives, processes for their preparation, pharmaceutical compositions comprising the derivatives, and methods of use thereof These compounds are anti-muscarinic agents with superior pharmacokinetic properties compared to existing drugs such as oxybutynin and tolterodine which are useful in the treatment of urinary incontinence, gastrointestinal hyperactivity (irritable bowel syndrome) and other smooth muscle contractile conditions. Among them, Fesoterodine, chemically 2-[(1R)-3-[bis(1-methylethyl)amino]-1-phenylpropyl]-4-hydroxymethylphenyl isobutyrate, is a new, potent and competitive muscarinic antagonist and useful in the potential treatment of urinary incontinence. Fesoterodine is represented by the following structural formula I:

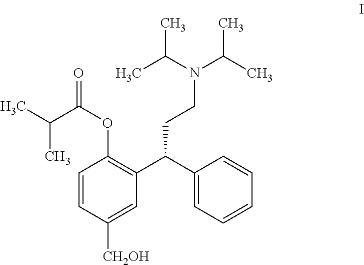

Processes for the preparation of fesoterodine and related compounds, and their pharmaceutically acceptable salts are disclosed in the U.S. Pat. Nos. 6,713,464 B1 and 6,858,650 B1; U.S. Patent Application No. 2006/0270738 and PCT Publication No. WO 2007/138440 A1.

According to the U.S. Pat. No. 6,713,464 B1 (herein after referred to as the '464 patent), fesoterodine is prepared by the reaction of (±)-6-bromo-4-phenylchroman-2-one with benzyl chloride in the presence of sodium iodide and anhydrous potassium carbonate in methanol and acetone to give (±)-3-(2-benzyloxy-5-bromophenyl)-3-phenylpropionic acid methyl ester as a light yellow oil, which by reduction with lithium aluminium hydride in tetrahydrofuran at room temperature, produces (±)-3-(2-benzyloxy-5-bromophenyl)-3-phenylpropan-1-ol. (±)-3-(2-Benzyloxy-5-bromophenyl)-3-phenyl propan-1-ol is then treated with p-toluenesulphonyl chloride in the presence of pyridine in dichloromethane to afford (±)-toluene-4-sulphonic acid 3-(2-benzyloxy-5-bromophenyl)-3-phenylpropyl ester. Reaction with N,N-diisopropylamine in acetonitrile at reflux temperature produces (±)-[3-(2-benzyloxy-5-bromophenyl)-3-phenylpropyl]-diisopropylamine as a brown and viscous syrup, which by resolution produces (R)-[3-(2-benzyloxy-5-bromophenyl)-3-phenylpropyl]-diisopropylamine, which is then subjected to Grignard reaction with ethylbromide and magnesium in the presence of solid carbon dioxide in tetrahydrofuran to produce (R)-4-benzyloxy-3-(3-diisopropylamino-1-phenylpropyl)-benzoic acid hydrochloride. Esterification with methanol in the presence of sulphuric acid produces (R)-4-benzyloxy-3-(3-diisopropylamino-1-phenylpropyl)-benzoic acid methyl ester, which is then reduced with lithium aluminium hydride to produce (R)-[4-benzyloxy-3-(3-diisopropylamino-1-phenylpropyl)-phenyl]-methanol, which is then subjected to deprotection with Raney-Nickel to produce (R)-2-(3-diisopropylamino-1-phenylpropyl)-4-hydroxymethylphenol, followed by condensation with isobutyryl chloride in an inert solvent in the presence of a base to give fesoterodine.

Fesoterodine obtained by the processes described in the above prior art does not have satisfactory purity for pharmaceutical use. Unacceptable amounts of impurities are generally formed along with fesoterodine. In addition, the processes involve the additional step of column chromatographic purifications or multiple crystallizations. Methods involving column chromatographic purifications are generally undesirable for large-scale operations as they require additional expensive setup adding to the cost of production, thereby making the processes commercially unfeasible.

It is known that synthetic compounds can contain extraneous compounds or impurities resulting from their synthesis or degradation. The impurities can be unreacted starting materials, by-products of the reaction, products of side reactions, or degradation products. Generally, impurities in an active pharmaceutical ingredient (API) may arise from degradation of the API itself, or during the preparation of the API. Impurities in fesoterodine or any active pharmaceutical ingredient (API) are undesirable and might be harmful.

Regulatory authorities worldwide require that drug manufactures isolate, identify and characterize the impurities in their products. Furthermore, it is required to control the levels of these impurities in the final drug compound obtained by the manufacturing process and to ensure that the impurity is present in the lowest possible levels, even if structural determination is not possible.

The product mixture of a chemical reaction is rarely a single compound with sufficient purity to comply with pharmaceutical standards. Side products and byproducts of the reaction and adjunct reagents used in the reaction will, in most cases, also be present in the product mixture. At certain stages during processing of the active pharmaceutical ingredient, the product must be analyzed for purity, typically, by HPLC, TLC or GC analysis, to determine if it is suitable for continued processing and, ultimately, for use in a pharmaceutical product. Purity standards are set with the intention of ensuring that an API is as free of impurities as possible, and, thus, are as safe as possible for clinical use. The United States Food and Drug Administration guidelines recommend that the amounts of some impurities limited to less than 0.1 percent.

Generally, impurities are identified spectroscopically and by other physical methods, and then the impurities are associated with a peak position in a chromatogram (or a spot on a TLC plate). Thereafter, the impurity can be identified by its position in the chromatogram, which is conventionally measured in minutes between injection of the sample on the column and elution of the particular component through the detector, known as the “retention time” (“Rt”). This time period varies daily based upon the condition of the instrumentation and many other factors. To mitigate the effect that such variations have upon accurate identification of an impurity, practitioners use “relative retention time” (“RRT”) to identify impurities. The RRT of an impurity is its retention time divided by the retention time of a reference marker.

It is known by those skilled in the art, the management of process impurities is greatly enhanced by understanding their chemical structures and synthetic pathways, and by identifying the parameters that influence the amount of impurities in the final product.

There is a need for pure fesoterodine or a pharmaceutically acceptable salt thereof substantially free of impurities with reduced particle size distribution, which has good flow properties, and better dissolution and solubility properties to obtain formulations with greater bioavailability.

### SUMMARY

In one aspect, provided herein is a dehydroxyfesoterodine compound, 2-[(1R)-3-[bis(1-methylethyl)amino]-1-phenylpropyl]-4-methylphenyl isobutyrate, having the following structural formula I(i):

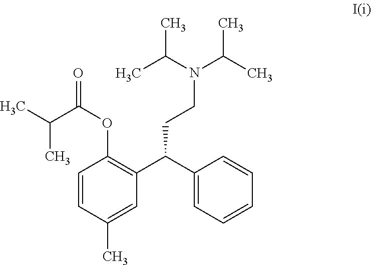

or its enantiomeric form or a mixture of enantiomeric forms thereof.

In another aspect, provided herein is an impurity of fesoterodine, fesoterodine dehydroxy impurity, 2-[(1R)-3-[bis(1-methylethyl)amino]-1-phenylpropyl]-4-methyl phenyl isobutyrate, of formula I(i).

In another aspect, encompassed herein is a process for synthesizing and isolating the dehydroxyfesoterodine.

In another aspect, provided herein is a highly pure fesoterodine or a pharmaceutically acceptable salt thereof substantially free of fesoterodine dehydroxy impurity.

In still further aspect, encompassed herein is a process for preparing the highly pure fesoterodine or a pharmaceutically acceptable salt thereof substantially free of dehydroxy impurity.

Exemplary pharmaceutically acceptable salts of fesoterodine include, but are not limited to, hydrochloride, hydrobromide, nitrate, sulfate, mandelate, oxalate, succinate, maleate, besylate, tosylate, palmitate, fumarate and tartarate; and more specifically fumarate.

In another aspect, encompassed herein is the use of pure (+)-N,N-diisopropyl-3-(2-hydroxy-5-hydroxymethylphenyl)-3-phenylpropylamine of formula II substantially free of dehydroxy compound of formula II(i) obtained by the process disclosed herein for preparing fesoterodine.

In another aspect, provided herein is a pharmaceutical composition comprising highly pure fesoterodine or a pharmaceutically acceptable salt thereof substantially free of dehydroxy impurity, and one or more pharmaceutically acceptable excipients.

In still another aspect, provided herein is a pharmaceutical composition comprising highly pure fesoterodine or a pharmaceutically acceptable salt thereof substantially free of dehydroxy impurity made by the process disclosed herein, and one or more pharmaceutically acceptable excipients.

In still further aspect, encompassed is a process for preparing a pharmaceutical formulation comprising combining highly pure fesoterodine or a pharmaceutically acceptable salt thereof substantially free of dehydroxy impurity with one or more pharmaceutically acceptable excipients.

In another aspect, provided herein is fesoterodine fumarate having a 90 volume-percent of the particles (D₉₀) with a size of less than or equal to about 200 microns, and specifically about 1 micron to about 190 microns.

In another aspect, encompassed herein is a process for preparing fesoterodine fumarate having a D₉₀ particle size of about 80 microns to about 200 microns, comprising providing a solution of fesoterodine fumarate in an alcohol solvent, combining the solution with an ether solvent, and isolating fesoterodine fumarate particles having a D₉₀ particle size of about 80 microns to about 200 microns under specific conditions.

In another aspect, the fesoterodine fumarate obtained by the process disclosed herein has a D₉₀ particle size of about 80 microns to about 190 microns, specifically about 85 microns to about 150 microns, and more specifically about 85 microns to about 120 microns.

In another aspect, encompassed herein is a process for controlling the particle size of fesoterodine fumarate substantially free of dehydroxy impurity, comprising:
a) providing solid particles of fesoterodine fumarate substantially free of dehydroxy impurity having a D₉₀ particle size of about 80 microns to about 200 microns; and
b) milling the solid particles of fesoterodine fumarate of step-(a) to obtain fesoterodine fumarate particles having a particle size which is suitable for homogeneous distribution of the drug substance in a tablet blend, in particular 90 volume-percent of the particles (D₉₀) have a size of about 1 micron to about 190 microns.

In another aspect, the highly pure fesoterodine fumarate substantially free of dehydroxy impurity disclosed herein for use in the pharmaceutical compositions has a 90 volume-percent of the particles (D₉₀) with a size of about 1 micron to about 200 microns, specifically about 5 microns to about 150 microns, more specifically about 10 microns to about 100 microns, and most specifically about 15 microns to about 60 microns.

In yet another aspect, provided herein is a pharmaceutical composition comprising fesoterodine fumarate having a D₉₀ particle size of about 1 micron to about 200 microns, and one or more pharmaceutically acceptable excipients.

In still another aspect, provided herein is a pharmaceutical composition comprising fesoterodine fumarate having a D₉₀ particle size of about 1 micron to about 200 microns made by the process disclosed herein, and one or more pharmaceutically acceptable excipients.

In still further aspect, encompassed is a process for preparing a pharmaceutical formulation comprising combining fesoterodine fumarate having a D₉₀ particle size of about 1 micron to about 200 microns with one or more pharmaceutically acceptable excipients.

### DETAILED DESCRIPTION

According to one aspect, there is provided a dehydroxyfesoterodine compound, 2-[(1R)-3-[bis(1-methylethyl)amino]-1-phenylpropyl]-4-methylphenyl isobutyrate, having the following structural formula I(i):

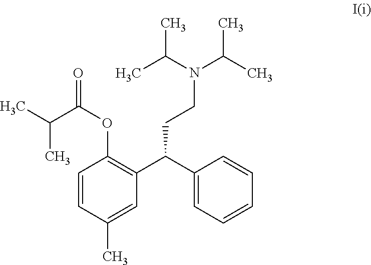

or an enantiomeric form or a mixture of enantiomeric forms thereof, or an acid addition salt thereof.

The acid addition salts of dehydroxyfesoterodine can be derived from a therapeutically acceptable acid such as hydrochloric acid, hydrobromic acid, sulfuric acid, nitric acid, phosphoric acid, acetic acid, propionic acid, oxalic acid, mandelic acid, succinic acid, maleic acid, fumaric acid, methanesulfonic acid, benzenesulfonic acid, toluenesulfonic acid, citric acid, and tartaric acid.

According to another aspect, there is provided an impurity of fesoterodine, fesoterodine dehydroxy impurity, 2-[(1R)-3-[bis(1-methylethyl)amino]-1-phenylpropyl]-4-methyl phenyl isobutyrate, of formula I(i).

The fesoterodine dehydroxy impurity is identified, isolated and synthesized. In one embodiment, the fesoterodine dehydroxy impurity can be used as a reference standard for determination of the purity of fesoterodine or a pharmaceutically acceptable salt thereof. The dehydroxy impurity is detected and resolved from fesoterodine by HPLC with an RRT of 1.8. The structure of the compound of formula I(i) is deduced with the aid of ¹H, ¹³C NMR & IR spectroscopy and FAB mass spectrometry. The parent ion at 395.57 is consistent with assigned structure.

The fesoterodine dehydroxy impurity has the following ¹H NMR (400 MHz, CDCl₃) δ(ppm): 0.98-1.03(d,3H), 1.31-1.37(d,3H), 2.19-2.32 (m,2H), 2.33(s,3H), 2.4-2.45(m,2H), 2.79-2.86(m,1H), 3.05-3.12(m,1H), 4.07-4.11(t,1H), 6.88-6.90(m,1H), 7.02-7.04(m,1H), 7.17-7.18(m,1H), 7.19-7.31(m,1H); MS: EI⁺ m/z (MH+): 396.7; and IR spectra on KBr having absorption bands at about 3028, 2969-2875, 1756, 1601-1496, 1470, 1387, 1128, 865, 737-700 cm⁻¹.

According to another aspect, there is provided an isolated fesoterodine dehydroxy impurity.

The present inventors have surprisingly found that the dehydroxy impurity is formed as an impurity during the synthesis of fesoterodine due to over reduction of (−)-N,N-diisopropyl-3-(2-benzyloxy-5-carbomethoxyphenyl)-3-phenylpropylamine intermediate.

The dehydroxy impurity is identified and isolated as follows: a) reducing (−)-N,N-diisopropyl-3-(2-benzyloxy-5-carbomethoxyphenyl)-3-phenylpropylamine of formula IV with lithium aluminium hydride to afford (+)-N,N-diisopropyl-3-(2-benzyloxy-5-hydroxymethylphenyl)-3-phenylpropylamine of formula III contaminated with the analogous dehydroxy compound of formula III(i); b) hydrogenating the compound of formula III obtained in step-(a) in the presence of a hydrogenation catalyst to produce (+)-N,N-diisopropyl-3-(2-hydroxy-5-hydroxymethylphenyl)-3-phenylpropyl amine of formula II contaminated with the analogous dehydroxy compound of formula II(i); c) condensing the compound of formula II obtained in step-(b) with isobutyryl chloride in the presence of triethylamine to produce crude fesoterodine of formula I contaminated with the corresponding dehydroxy impurity of formula I(i); d) subjecting the crude fesoterodine of step-(c) to column chromatography and eluting with a gradient mobile phase to produce an eluent containing the dehydroxy impurity of formula I(i); and e) isolating the dehydroxy impurity from the eluent. The formation of the dehydroxy impurity is shown in the following scheme:

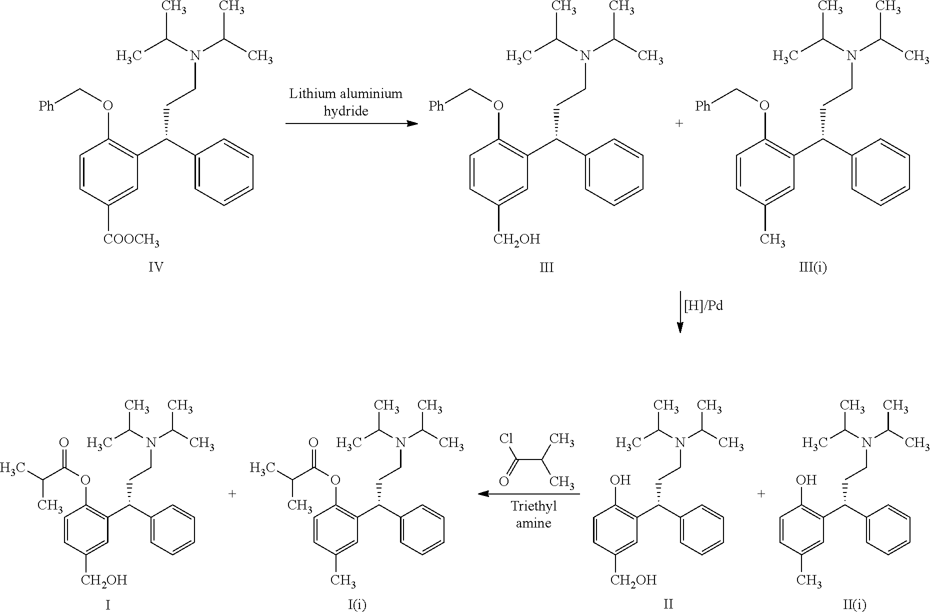

Extensive experimentation was carried out by the present inventors to reduce the level of the dehydroxy impurity in fesoterodine. As a result, it has been found that the dehydroxy impurity formed in the preparation of the fesoterodine can be reduced or completely removed by providing a solution of the (+)-N,N-diisopropyl-3-(2-hydroxy-5-hydroxymethyl phenyl)-3-phenylpropylamine of formula II contaminated with the analogous dehydroxy compound of formula II(i) in a solvent, contacting the solution with an anti-solvent to form a precipitate, recovering pure (+)-N,N-diisopropyl-3-(2-hydroxy-5-hydroxymethyl phenyl)-3-phenylpropylamine of formula II substantially free of dehydroxy compound of formula II(i), and then converting the pure intermediate to fesoterodine or a pharmaceutically acceptable salt thereof substantially free of dehydroxy impurity of formula I(i).

In addition to the presence of impurities, the solid state physical properties of an active pharmaceutical ingredient (API), such as fesoterodine fumarate, can be very important in formulating a drug substance, and can have profound effects on the ease and reproducibility of formulation. Particle size, for example, may affect the flowability and mixability of a drug substance. In cases, where the active ingredient has good flow properties, tablets can be prepared by direct compression of the ingredients. However, in many cases the particle size of the active substance is very small, the active substance is cohesive or has poor flow properties. Small particles are also filtered and washed more slowly during isolation processes, and thus may increase the time and expense of manufacturing a drug formulation.

Fesoterodine fumarate is a white to off-white powder and it is freely soluble in aqueous solvents, soluble in some polar protic organic solvents (such as ethanol, methanol, glacial acetic acid, 2-propanol, propylene glycol) and polar non protic solvents (such as acetone, DMF, DMSO, acetonitrile), slightly soluble in toluene and it is practically insoluble in heptane.

According to another aspect, there is provided a highly pure fesoterodine or a pharmaceutically acceptable salt thereof substantially free of fesoterodine dehydroxy impurity.

As used herein, “highly pure fesoterodine or a pharmaceutically acceptable salt thereof substantially free of dehydroxy impurity” refers to fesoterodine or a pharmaceutically acceptable salt thereof comprising dehydroxy impurity or its enantiomeric form or a mixture of enantiomeric forms thereof in an amount of less than about 0.2 area-% as measured by HPLC. Specifically, the fesoterodine, as disclosed herein, contains less than about 0.15 area-%, more specifically less than about 0.05 area-%, still more specifically less than about 0.02 area-% of dehydroxy impurity, and most specifically is essentially free of dehydroxy impurity.

In one embodiment, the highly pure fesoterodine or a pharmaceutically acceptable salt thereof disclosed herein comprises a fesoterodine dehydroxy impurity in an amount of about 0.01 area-% to about 0.15 area-%, specifically in an amount of about 0.01 area-% to about 0.05 area-%, as measured by HPLC.

In another embodiment, the highly pure fesoterodine or a pharmaceutically acceptable salt thereof disclosed herein has a total purity of greater than about 98%, specifically greater than about 99%, more specifically greater than about 99.5%, and most specifically greater than about 99.9% as measured by HPLC. For example, the purity of the highly pure fesoterodine or a pharmaceutically acceptable salt thereof is about 98% to about 99.9%, or about 99% to about 99.99%.

In another embodiment, the highly pure fesoterodine or a pharmaceutically acceptable salt thereof disclosed herein is essentially free of fesoterodine dehydroxy impurity.

The term “fesoterodine or a pharmaceutically acceptable salt thereof essentially free of fesoterodine dehydroxy impurity” refers to fesoterodine or a pharmaceutically acceptable salt thereof contains a non-detectable amount of fesoterodine dehydroxy impurity as measured by HPLC.

Exemplary pharmaceutically acceptable salts of fesoterodine include, but are not limited to, hydrochloride, hydrobromide, nitrate, sulfate, mandelate, oxalate, succinate, maleate, besylate, tosylate, palmitate, fumarate and tartarate; and more specifically fumarate.

According to another aspect, there is provided a process for preparing pure fesoterodine or a pharmaceutically acceptable salt thereof substantially free of dehydroxy impurity, comprising:
a) providing a solution of crude (+)-N,N-diisopropyl-3-(2-hydroxy-5-hydroxymethyl phenyl)-3-phenylpropylamine of formula II in a first solvent;
b) optionally, subjecting the solution obtained in step-(a) to carbon treatment or silica gel treatment;
c) admixing the solution with an anti-solvent to form a precipitate;
d) recovering pure (+)-N,N-diisopropyl-3-(2-hydroxy-5-hydroxymethylphenyl)-3-phenyl propylamine of formula II substantially free of dehydroxy impurity of formula II(i) from the precipitate;
e) condensing the pure compound of formula II obtained in step-(d) with isobutyryl chloride in a second solvent, optionally in the presence of a base, to produce pure fesoterodine of formula I substantially free of dehydroxy impurity of formula I(i); and
f) optionally, converting the pure fesoterodine base obtained in step-(e) into a pharmaceutically acceptable acid addition salt thereof; or
g) optionally, converting the pure fesoterodine base obtained in step-(e) into its mandelate salt and then converting the fesoterodine mandelate salt formed into a pharmaceutically acceptable acid addition salt of fesoterodine.

As used herein, “pure (+)-N,N-diisopropyl-3-(2-hydroxy-5-hydroxy methylphenyl)-3-phenylpropylamine substantially free of dehydroxy impurity” refers to (+)-N,N-diisopropyl-3-(2-hydroxy-5-hydroxymethylphenyl)-3-phenylpropyl amine comprising dehydroxy impurity of formula II(i) in an amount of less than about 0.2 area-% as measured by HPLC.

The first solvent used in step-(a) is selected from the group consisting of water, an alcohol, an ester, acetone, acetonitrile, N,N-dimethylformamide, N,N-dimethylacetamide, dimethylsulfoxide, and mixtures thereof.

Exemplary alcohol solvents include, but are not limited to, C₁ to C₆ straight or branched chain alcohol solvents such as methanol, ethanol, n-propanol, isopropyl alcohol, isobutanol, n-butanol, tert-butanol, amyl alcohol, isoamyl alcohol, hexanol, and mixtures thereof. Specific alcohol solvents are methanol, ethanol, isopropyl alcohol, and mixtures thereof.

Exemplary ester solvents include, but are not limited to, ethyl acetate, isopropyl acetate, n-butyl acetate, tert-butyl acetate, and the like and mixtures thereof. A specific ester solvent is ethyl acetate.

Specifically, the first solvent is selected from the group consisting of methanol, ethanol, isopropyl alcohol, ethyl acetate, acetonitrile, N,N-dimethylformamide, N,N-dimethylacetamide, dimethylsulfoxide, and mixtures thereof; and more specifically, isopropyl alcohol, ethyl acetate, and mixtures thereof.

Step-(a) of providing a solution of crude (+)-N,N-diisopropyl-3-(2-hydroxy-5-hydroxymethylphenyl)-3-phenylpropylamine of formula II includes dissolving crude (+)-N,N-diisopropyl-3-(2-hydroxy-5-hydroxymethylphenyl)-3-phenylpropylamine in the first solvent, or obtaining an existing solution from a previous processing step.

In one embodiment, the crude (+)-N,N-diisopropyl-3-(2-hydroxy-5-hydroxymethyl phenyl)-3-phenylpropylamine of formula II is dissolved in the first solvent at a temperature of about 25° C. to the reflux temperature of the solvent used, specifically at about 25° C. to about 110° C., and more specifically at about 30° C. to about 80° C.

As used herein, “reflux temperature” means the temperature at which the solvent or solvent system refluxes or boils at atmospheric pressure.

In another embodiment, the solution in step-(a) is prepared by reducing (−)-N,N-diisopropyl-3-(2-benzyloxy-5-carbomethoxyphenyl)-3-phenylpropylamine of formula IV with lithium aluminium hydride, optionally in the presence of an organic or inorganic base, in a solvent under conditions to produce crude (+)-N,N-diisopropyl-3-(2-benzyloxy-5-hydroxymethylphenyl)-3-phenylpropylamine of formula III, which is then hydrogenated in the presence of a hydrogenation catalyst in a solvent to produce a reaction mass containing crude (+)-N,N-diisopropyl-3-(2-hydroxy-5-hydroxymethylphenyl)-3-phenylpropylamine of formula II followed by usual work-up such as washings, extractions, evaporations or a combination thereof. In one embodiment, the work-up includes dissolving or extracting the resulting crude (+)-N,N-diisopropyl-3-(2-hydroxy-5-hydroxymethylphenyl)-3-phenylpropylamine of formula II in the first solvent at a temperature of about 25° C. to the reflux temperature of the solvent used, specifically at about 25° C. to about 110° C., and more specifically at about 30° C. to about 80° C.

The carbon treatment or silica gel treatment in step-(b) is carried out by methods known in the art, for example, by stirring the solution with finely powdered carbon or silica gel at a temperature of below about 70° C. for at least 15 minutes, specifically at a temperature of about 40° C. to about 70° C. for at least 30 minutes; and filtering the resulting mixture through a filtration bed such as hyflo to obtain a filtrate containing the compound of formula II by removing charcoal or silica gel. Specifically, the finely powdered carbon is an active carbon. A specific mesh size of silica gel is 40-500 mesh, and more specifically 60-120 mesh.

The anti-solvent used in step-(c) is selected from the group consisting of an ether, a hydrocarbon, and mixtures thereof. Exemplary ether solvents include, but are not limited to, tetrahydrofuran, dioxane, diethyl ether, diisopropyl ether, monoglyme, diglyme, and the like, and mixtures thereof. Exemplary hydrocarbon solvents include, but are not limited to, n-pentane, n-hexane, n-heptane and their isomers, petroleum ether, cyclohexane, toluene, xylene, and mixtures thereof. Specifically, the anti-solvent is selected from the group consisting of diisopropyl ether, diethyl ether, n-hexane, petroleum ether, and mixtures thereof.

As used herein, “anti-solvent” means a solvent which when added to an existing solution of a substance reduces the solubility of the substance.

Admixing of the solution with anti-solvent in step-(c) is done in a suitable order, for example, the anti-solvent is added to the solution, or alternatively, the solution is added to the anti-solvent. The addition is, for example, carried out drop wise or in one portion or in more than one portion. The addition is specifically carried out at a temperature of about 20° C. to about 80° C. for at least 20 minutes, and more specifically at a temperature of about 30° C. to about 75° C. for about 30 minutes to about 4 hours. After completion of addition process, the resulting mass is cooled and followed by stirring at a temperature of below 30° C. for at least 10 minutes, and more specifically at about 0° C. to about 30° C. for about 30 minutes to about 10 hours.

The recovering in step-(d) is carried out by methods such as filtration, filtration under vacuum, decantation, centrifugation, or a combination thereof. In one embodiment, the pure (+)-N,N-diisopropyl-3-(2-hydroxy-5-hydroxymethylphenyl)-3-phenyl propylamine of formula II substantially free of dehydroxy compound of formula II(i) is recovered by filtration employing a filtration media of, for example, a silica gel or celite.

The pure (+)-N,N-diisopropyl-3-(2-hydroxy-5-hydroxymethylphenyl)-3-phenyl propylamine of formula II substantially free of dehydroxy compound of formula II(i) obtained by above process may be further dried in, for example, a Vacuum Tray Dryer, a Rotocon Vacuum Dryer, a Vacuum Paddle Dryer or a pilot plant Rota vapor, to further lower residual solvents.

In one embodiment, the drying is carried out at atmospheric pressure or reduced pressures, such as below about 200 mm Hg, or below about 50 mm Hg, at temperatures such as about 35° C. to about 75° C. The drying can be carried out for any desired time period that achieves the desired result, such as about 1 to 20 hours. Drying may also be carried out for shorter or longer periods of time depending on the product specifications. Temperatures and pressures will be chosen based on the volatility of the solvent being used and the foregoing should be considered as only a general guidance. Drying can be suitably carried out in a tray dryer, vacuum oven, air oven, or using a fluidized bed drier, spin flash dryer, flash dryer and the like. Drying equipment selection is well within the ordinary skill in the art.

The condensation reaction in step-(e) can be carried out by the methods known in the art. The reaction is specifically carried out at a temperature of below about 50° C., more specifically at a temperature of about −20° C. to about 30° C. for at least 20 minutes, and still more specifically at a temperature of about −15° C. to about 15° C. for about 30 minutes to about 4 hours. Exemplary second solvent used in step-(e) includes, but is not limited to, a hydrocarbon, a chlorinated hydrocarbon, a nitrile, an ester, an ether, and mixtures thereof. A specific second solvent is methylene chloride.

The base used in step-(e) is an organic or inorganic base. In one embodiment, the base is an organic base. Specific organic bases are organic amine bases of formula NR₁R₂R₃ wherein R₁, R₂ and R₃ are each independently hydrogen, C₁₋₆ straight or branched chain alkyl, aryl alkyl, C₃₋₁₀ single or fused ring optionally substituted, alkylcycloalkyls or independently R₁, R₂ and R₃ combine with each other to form C₃₋₇ membered cycloalkyl ring or heterocyclic system containing one or more heteroatom. A specific organic base is triethylamine.

Exemplary inorganic bases include, but are not limited to, hydroxides, carbonates, alkoxides and bicarbonates of alkali or alkaline earth metals. Specific inorganic bases are sodium hydroxide, calcium hydroxide, magnesium hydroxide, potassium hydroxide, lithium hydroxide, sodium carbonate, potassium carbonate, lithium carbonate, sodium tert-butoxide, sodium isopropoxide and potassium tert-butoxide, and more specifically sodium hydroxide, potassium hydroxide, sodium carbonate and potassium carbonate.

Pharmaceutically acceptable salts of fesoterodine can be prepared in high purity by using the pure fesoterodine substantially free of dehydroxy impurity obtained in step-(e), by known methods.

The conversion of fesoterodine base to fesoterodine mandelate in step-(g) is carried out by providing a solution of fesoterodine free base in an alcoholic solvent, followed by the addition of mandelic acid to the solution and then isolating pure fesoterodine mandelate substantially free of dehydroxy impurity. The pure fesoterodine mandelate obtained is further converted to a pharmaceutically acceptable acid addition salt of fesoterodine by treating the fesoterodine mandelate with a base in an organic solvent to liberate fesoterodine free base and then converting the fesoterodine free base into its pharmaceutically acceptable salts thereof. A preferable pharmaceutically acceptable salt of fesoterodine is fesoterodine fumarate. The base used is an organic or inorganic base selected from the group as described above. Preferably the base is an inorganic base. Exemplary organic solvents include, but are not limited to, an alcohol, a ketone, a hydrocarbon, a chlorinated hydrocarbon solvent, and mixtures thereof.

In another embodiment, the pure fesoterodine or a pharmaceutically acceptable salt thereof substantially free of dehydroxy impurity of formula I(i) is prepared analogously, by purifying crude fesoterodine or its mandelate salt as per the processes described herein above, for example, by: a) providing a solution of crude fesoterodine or its mandelate salt thereof in a solvent selected from the group consisting of water, an alcohol, an ester, acetone, acetonitrile, N,N-dimethylformamide, N,N-dimethylacetamide, dimethylsulfoxide, and mixtures thereof; b) optionally, subjecting the solution obtained in step-(a) to carbon treatment or silica gel treatment; c) admixing the solution with an anti-solvent to produce a reaction mass; and d) recovering pure fesoterodine of formula I or its mandelate salt substantially free of dehydroxy impurity of formula I(i) from the reaction mass, and optionally converting the pure fesoterodine or its mandelate formed into a pharmaceutically acceptable acid addition salt thereof.

The term “crude fesoterodine or fesoterodine mandelate” as used herein refers to fesoterodine or fesoterodine mandelate containing greater than about 0.2 area-%, specifically greater than about 0.3 area-%, more specifically greater than about 1 area-% and most specifically greater than about 3 area-% of the dehydroxy impurity of formula I(i).

Pharmaceutically acceptable salts of fesoterodine can be prepared in high purity by using the substantially pure fesoterodine free base or its mandelate salt substantially free of dehydroxy impurity obtained by the methods disclosed herein, by known methods.

According to another aspect, there is provided a process for preparing dehydroxy fesoterodine, 2-[(1R)-3-[bis(1-methylethyl)amino]-1-phenylpropyl]-4-methylphenyl isobutyrate, of formula I(i):

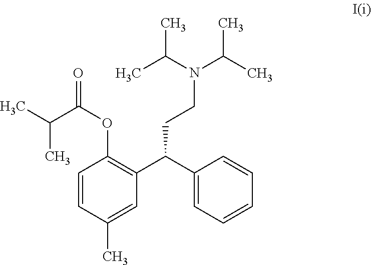

comprising condensing (+)-N,N-diisopropyl-3-(2-hydroxy-5-methylphenyl)-3-phenyl propylamine (tolterodine) of formula II(i):

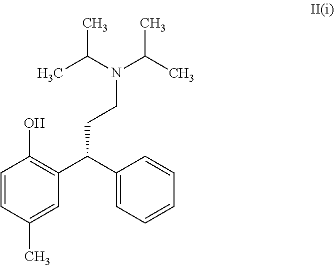

with isobutyryl chloride, optionally in the presence of a base, in a suitable solvent to provide the 2-[(1R)-3-[bis(1-methylethyl)amino]-1-phenylpropyl]-4-methylphenyl isobutyrate (dehydroxy fesoterodine or fesoterodine dehydroxy impurity) of formula I(i).

Exemplary solvents used for the condensation include, but are not limited to, an alcohol, a ketone, a cyclic ether, an aliphatic ether, a hydrocarbon, a chlorinated hydrocarbon, a nitrile, an ester and the like, and mixtures thereof.

Exemplary alcohol solvents include, but are not limited to, C₁ to C₈ straight or branched chain alcohol solvents such as methanol, ethanol, propanol, butanol, amyl alcohol, hexanol, and mixtures thereof. Specific alcohol solvents are methanol, ethanol, isopropyl alcohol, and mixtures thereof, and most specific alcohol solvent is isopropyl alcohol. Exemplary ketone solvents include, but are not limited to, acetone, methyl isobutyl ketone, and the like, and mixtures thereof. Exemplary cyclic ether solvents include, but are not limited to, tetrahydrofuran, dioxane, and the like, and mixtures thereof. Exemplary nitrile solvents include, but are not limited to, acetonitrile and the like, and mixtures thereof. Exemplary ester solvents include, but are not limited to, ethyl acetate, isopropyl acetate, and the like and mixtures thereof. Exemplary hydrocarbon solvents include, but are not limited to, n-pentane, n-hexane and n-heptane and isomers or mixtures thereof, cyclohexane, toluene and xylene. Specific hydrocarbon solvent is toluene. Exemplary chlorinated hydrocarbon solvents include, but are not limited to, methylene chloride, ethyl dichloride, chloroform, carbon tetrachloride and mixtures thereof. Specific chlorinated hydrocarbon solvent is methylene chloride.

Specific solvents are n-hexane, n-heptane, cyclohexane, toluene, xylene, methylene chloride, ethyl dichloride, chloroform, and mixtures thereof; and most specifically methylene chloride.

In one embodiment, the reaction is carried out at a temperature of below about 50° C., specifically at a temperature of about −20° C. to about 30° C. for at least 20 minutes, and still more specifically at a temperature of about −15° C. to about 15° C. for about 30 minutes to about 4 hours.

The base used in the condensation is an organic or inorganic base as described above.

In one embodiment, the compound of formula I(i) obtained is isolated using an organic solvent by methods such as substantially complete evaporation of the solvent, concentrating the solution or distillation of solvent under inert atmosphere, cooling, partial removal of the solvent from the solution, addition of a precipitating solvent, or a combination thereof. Exemplary solvents include, but are not limited to, an alcohol, a hydrocarbon, a ketone, a cyclic ether, an aliphatic ether, a nitrile, and the like, and mixtures thereof.

The (+)-N,N-Diisopropyl-3-(2-hydroxy-5-methylphenyl)-3-phenylpropylamine of formula II(i) (tolterodine) used as starting material may be obtained by processes described in the prior art.

Further encompassed herein is the use of the highly pure fesoterodine or a pharmaceutically acceptable salt thereof, specifically fesoterodine fumarate, substantially free of dehydroxy impurity for the manufacture of a pharmaceutical composition together with a pharmaceutically acceptable carrier.

A specific pharmaceutical composition of highly pure fesoterodine or a pharmaceutically acceptable salt thereof substantially free of dehydroxy impurity is selected from a solid dosage form and an oral suspension.

According to another aspect, there is provided a method for treating a patient suffering from diseases caused by overactive bladder with symptoms of urge urinary incontinence, urgency, and frequency; comprising administering a therapeutically effective amount of the highly pure fesoterodine or a pharmaceutically acceptable salt thereof, specifically fesoterodine fumarate, substantially free of dehydroxy impurity, or a pharmaceutical composition that comprises a therapeutically effective amount of highly pure fesoterodine or a pharmaceutically acceptable salt thereof substantially free of dehydroxy impurity, along with pharmaceutically acceptable excipients.

According to another aspect, there is provided pharmaceutical compositions comprising highly pure fesoterodine or a pharmaceutically acceptable salt thereof, specifically fesoterodine fumarate, substantially free of dehydroxy impurity prepared according to processes disclosed herein and one or more pharmaceutically acceptable excipients.

According to another aspect, there is provided a process for preparing a pharmaceutical formulation comprising combining highly pure fesoterodine or a pharmaceutically acceptable salt thereof, specifically fesoterodine fumarate, substantially free of dehydroxy impurity prepared according to processes disclosed herein, with one or more pharmaceutically acceptable excipients.

According to another aspect, there is provided fesoterodine fumarate having a 90 volume-percent of the particles (D₉₀) with a size of less than or equal to about 200 microns.

In one embodiment, the fesoterodine fumarate disclosed herein has a D₉₀ particle size of about 1 micron to about 190 microns.

According to another aspect, there is provided a process for the preparation of fesoterodine fumarate substantially free of dehydroxy impurity having a D₉₀ particle size of about 80 microns to about 200 microns, comprising:
a) providing a solution of fesoterodine fumarate in an alcohol solvent;
b) optionally, filtering the solvent solution to remove extraneous matter;
c) optionally, seeding the solution;
d) admixing the solution with an anti-solvent to produce a reaction mass; and
e) recovering fesoterodine fumarate particles substantially free of dehydroxy impurity having a D₉₀ particle size of about 80 microns to about 200 microns from the reaction mass obtained in step-(d).

The process can produce fesoterodine fumarate crystalline particles in substantially pure form.

Fesoterodine fumarate having the desired particle size, obtained by the process disclosed herein, can be filtered off and dried easily. The process disclosed herein allows the dissolution rate of the fesoterodine fumarate to be controlled. Processing fesoterodine fumarate to bring the particle size within a particular range can also enhance manufacturing capability, allowing the preparation of pharmaceutical compositions that exhibit an improved bioavailability of fesoterodine fumarate. Fesoterodine fumarate of the present invention is thus well suited for formulations.

The fesoterodine fumarate obtained by the process described hereinabove is stable, consistently reproducible and has good flow properties, and which is particularly suitable for bulk preparation and handling, and so, the fesoterodine fumarate particles substantially free of dehydroxy impurity having a D₉₀ particle size of about 80 microns to about 200 microns is suitable for formulating fesoterodine fumarate.

In one embodiment, the fesoterodine fumarate obtained by the process disclosed herein remains in the same crystalline form and stable, when stored at a temperature of about 25±2° C. and at a relative humidity of about 60±5% for a period of at least one month.

In still another embodiment, the fesoterodine fumarate obtained by the process disclosed herein remains in the same crystalline form and stable, when stored at a temperature of about 25±2° C. and at a relative humidity of about 60±5% for a period of 3 months.

The term “remains stable”, as defined herein, refers to lack of formation of impurities, while being stored as described hereinbefore.

Exemplary alcohol solvents used in step-(a) include, but are not limited to, C₁ to C₆ straight or branched chain alcohol solvents such as methanol, ethanol, n-propanol, isopropyl alcohol, n-butanol, tert-butanol, amyl alcohol, hexanol, and mixtures thereof. Specific alcohol solvents are methanol, ethanol, isopropyl alcohol and mixtures thereof, and more specifically isopropyl alcohol.

Step-(a) of providing a solution of fesoterodine fumarate includes dissolving fesoterodine fumarate in the alcoholic solvent, or obtaining an existing solution from a previous processing step.

In one embodiment, the fesoterodine fumarate is dissolved in the alcohol solvent at a temperature of above about 50° C., specifically at a temperature of about 50° C. to the reflux temperature of the solvent used, and more specifically at the reflux temperature of the solvent used.

In another embodiment, the solution in step-(a) is prepared by providing a solution of fesoterodine free base in the alcoholic solvent, admixing fumaric acid with the solution followed by heating the mass at a temperature of above about 50° C. to form a clear solution.

In one embodiment, the solution of fesoterodine free base is provided by dissolving the fesoterodine free base in the alcoholic solvent under stirring at a temperature of below about reflux temperature of the solvent used, specifically at about 20° C. to about 80° C., and more specifically at about 20° C. to about 60° C. In another embodiment, the solution of fesoterodine free base is prepared by condensing the pure (+)-N,N-diisopropyl-3-(2-hydroxy-5-hydroxymethylphenyl)-3-phenylpropylamine of formula II substantially free of dehydroxy compound of formula II(i) with isobutyryl chloride in a suitable solvent, optionally in the presence of a base, to produce a reaction mass containing fesoterodine free base followed by usual work-up such as washings, extractions, evaporations or a combination thereof. In one embodiment, the work-up also includes dissolving or extracting the resulting fesoterodine free base in the alcoholic solvent under stirring at a temperature of below about reflux temperature of the solvent used, specifically at about 20° C. to about 80° C., and more specifically at about 20° C. to about 60° C.

In yet another embodiment, the solution of fesoterodine free base is prepared by treating an acid addition salt of fesoterodine with a base to liberate fesoterodine free base, followed by dissolving or extracting the fesoterodine free base in the alcoholic solvent at a temperature of below about reflux temperature of the solvent used, specifically at about 20° C. to about 80° C., and more specifically at about 20° C. to about 60° C.

In another embodiment, the acid addition salt of fesoterodine is derived from a therapeutically acceptable acid such as mandelic acid, hydrochloric acid, hydrobromic acid, acetic acid, propionic acid, sulfuric acid, nitric acid, phosphoric acid, succinic acid, maleic acid, fumaric acid, citric acid, glutaric acid, citraconic acid, glutaconic acid, tartaric acid, malic acid, and ascorbic acid. Specific acid addition salts are fesoterodine fumarate and fesoterodine mandelate.

The treatment of an acid addition salt with a base is carried out in a solvent. A wide variety of solvents such as chlorinated solvents, alcohols, ketones, hydrocarbon solvents, esters, ether solvents etc., can be used.

In one embodiment, the base is an organic or inorganic base selected from the group as described above.

The solution obtained in step-(a) may optionally be subjected to carbon treatment or silica gel treatment. The carbon treatment or silica gel treatment is carried out by methods as described above.

Admixing in step-(d) is done in a suitable order, for example, the solution is added to the anti-solvent, or alternatively, the anti-solvent is added to the solution. The addition is, for example, carried out drop wise or in one portion or in more than one portion. The addition is specifically carried out at a temperature of below 50° C. for at least 15 minutes, and more specifically at a temperature of about 15° C. to about 40° C. for about 20 minutes to about 2 hours. After completion of addition process, the resulting mass is specifically stirred for at least 2 hours, more specifically for about 4 hours to about 22 hours, and most specifically for about 5 hours to about 18 hours, at a temperature of about 20° C. to about 40° C.

The anti-solvent is an ether solvent. Exemplary ether solvents include, but are not limited to, diisopropyl ether, diethyl ether, tetrahydrofuran, dioxane, monoglyme, diglyme and the like, and mixtures thereof. Specific anti-solvents are diisopropyl ether, diethyl ether, and mixtures thereof; and most specifically diisopropyl ether.

The recovering in step-(e) is carried out by methods such as filtration, filtration under vacuum, decantation, centrifugation, or a combination thereof. In one embodiment, the fesoterodine fumarate is recovered by filtration employing a filtration media of, for example, a silica gel or celite.

The pure fesoterodine fumarate has a D₉₀ particle size of about 80 microns to about 200 microns obtained by the above process may be further dried in, for example, a Vacuum Tray Dryer, a Rotocon Vacuum Dryer, a Vacuum Paddle Dryer or a pilot plant Rota vapor, to further lower residual solvents. Drying can be carried out under reduced pressure until the residual solvent content reduces to the desired amount such as an amount that is within the limits given by the International Conference on Harmonization of Technical Requirements for Registration of Pharmaceuticals for Human Use (“ICH”) guidelines.

In one embodiment, the drying is carried out at atmospheric pressure or reduced pressures, such as below about 200 mm Hg, or below about 50 mm Hg, at temperatures such as about 35° C. to about 70° C. The drying can be carried out for any desired time period that achieves the desired result, such as times about 1 to 20 hours. Drying may also be carried out for shorter or longer periods of time depending on the product specifications. Temperatures and pressures will be chosen based on the volatility of the solvent being used and the foregoing should be considered as only a general guidance. Drying can be suitably carried out in a tray dryer, vacuum oven, air oven, or using a fluidized bed drier, spin flash dryer, flash dryer, and the like. Drying equipment selection is well within the ordinary skill in the art.

Fesoterodine fumarate particles obtained by the process disclosed herein have good flow properties and having a particle size which is suitable for homogeneous distribution of the drug substance in a tablet blend.

In one embodiment, the fesoterodine fumarate obtained by the process disclosed herein above has a D₉₀ particle size of about 80 microns to about 190 microns, specifically about 85 microns to about 150 microns, and more specifically about 85 microns to about 120 microns.

In another embodiment, the particle sizes of the fesoterodine fumarate, obtained by the process disclosed herein, can be further reduced by a mechanical process of reducing the size of particles which includes any one or more of cutting, chipping, crushing, milling, grinding, micronizing, trituration or other particle size reduction methods known in the art, to bring the fesoterodine fumarate to the desired particle size range which is suitable for homogeneous distribution of the drug substance in a tablet blend.

In another embodiment, the fesoterodine fumarate having a D₉₀ particle size of about 80 microns to about 200 microns is milled to fesoterodine fumarate having smaller particle size in a milling process that is adapted to the desired particle size. Thus, the milling process provides control over the obtained particle size of fesoterodine fumarate. For example, milling can be performed by a cone mill, which operates by breaking particles with an impeller that revolves within a conical perforated screen.

According to another aspect, there is provided a process for controlling the particle size of fesoterodine fumarate, comprising:
a) providing solid particles of fesoterodine fumarate having a D₉₀ particle size of about 80 microns to about 200 microns; and
b) milling the fesoterodine fumarate of step-(a) to obtain fesoterodine fumarate having a D₉₀ particle size of about 1 micron to about 190 microns.

According to another aspect, there is provided a process for producing fesoterodine fumarate having a D₉₀ particle size of about 1 micron to about 190 microns comprising:
a) providing a solution of fesoterodine fumarate in an alcohol solvent;
b) optionally, filtering the solvent solution to remove extraneous matter;
c) optionally, seeding the solution;
d) admixing the solution with an anti-solvent to produce a reaction mass;
e) recovering fesoterodine fumarate particles substantially free of dehydroxy impurity having a D₉₀ particle size of about 80 microns to about 200 microns from the reaction mass obtained in step-(d); and
f) milling the crystalline fesoterodine fumarate obtained in step-(e) to obtain fesoterodine fumarate having a D₉₀ particle size of about 1 micron to about 190 microns.

In one embodiment, the highly pure fesoterodine fumarate substantially free of dehydroxy impurity disclosed herein for use in the pharmaceutical compositions has a 90 volume-percent of the particles (D₉₀) having a size of about 1 micron to about 200 microns, specifically about 5 microns to about 150 microns, more specifically about 10 microns to about 100 microns, and most specifically about 15 microns to about 60 microns.

According to another aspect, there is provided a pharmaceutical composition comprising fesoterodine fumarate having a D₉₀ particle size of about 1 micron to about 200 microns, and one or more pharmaceutically acceptable excipients.

According to another aspect, there is provided a pharmaceutical composition comprising fesoterodine fumarate having a D₉₀ particle size of about 1 micron to about 200 microns made by the process disclosed herein, and one or more pharmaceutically acceptable excipients.

According to another aspect, there is provided a process for preparing a pharmaceutical formulation comprising combining fesoterodine fumarate having a D₉₀ particle size of about 1 micron to about 200 microns with one or more pharmaceutically acceptable excipients.

Yet in another embodiment, pharmaceutical compositions comprise at least a therapeutically effective amount of highly pure fesoterodine or a pharmaceutically acceptable salt thereof substantially free of dehydroxy impurity. In one embodiment, the pharmaceutically acceptable salt of fesoterodine includes fesoterodine fumarate and more specifically fesoterodine fumarate having a D₉₀ particle size of about 1 micron to about 200 microns obtained by the processes disclosed herein. Such pharmaceutical compositions may be administered to a mammalian patient in a dosage form, e.g., solid, liquid, powder, elixir, aerosol, syrups, injectable solution, etc. Dosage forms may be adapted for administration to the patient by oral, buccal, parenteral, ophthalmic, rectal and transdermal routes or any other acceptable route of administration. Oral dosage forms include, but are not limited to, tablets, pills, capsules, syrup, troches, sachets, suspensions, powders, lozenges, elixirs and the like. The highly pure fesoterodine fumarate substantially free of dehydroxy impurity may also be administered as suppositories, ophthalmic ointments and suspensions, and parenteral suspensions, which are administered by other routes.

The pharmaceutical compositions further contain one or more pharmaceutically acceptable excipients. Suitable excipients and the amounts to use may be readily determined by the formulation scientist based upon experience and consideration of standard procedures and reference works in the field, e.g., the buffering agents, sweetening agents, binders, diluents, fillers, lubricants, wetting agents and disintegrants described hereinabove.

In one embodiment, capsule dosage forms contain highly pure fesoterodine fumarate substantially free of dehydroxy impurity within a capsule which may be coated with gelatin. Tablets and powders may also be coated with an enteric coating. Suitable enteric coating agents include phthalic acid cellulose acetate, hydroxypropylmethyl cellulose phthalate, polyvinyl alcohol phthalate, carboxy methyl ethyl cellulose, a copolymer of styrene and maleic acid, a copolymer of methacrylic acid and methyl methacrylate, and like materials, and if desired, the coating agents may be employed with suitable plasticizers and/or extending agents. A coated capsule or tablet may have a coating on the surface thereof or may be a capsule or tablet comprising a powder or granules with an enteric-coating.

Tableting compositions may have few or many components depending upon the tableting method used, the release rate desired and other factors. For example, the compositions described herein may contain diluents such as cellulose-derived materials like powdered cellulose, microcrystalline cellulose, microfine cellulose, methyl cellulose, ethyl cellulose, hydroxyethyl cellulose, hydroxypropyl cellulose, hydroxypropylmethyl cellulose, carboxymethyl cellulose salts and other substituted and unsubstituted celluloses; starch; pregelatinized starch; inorganic diluents such calcium carbonate and calcium diphosphate and other diluents known to one of ordinary skill in the art. Yet other suitable diluents include waxes, sugars (e.g. lactose) and sugar alcohols such as mannitol and sorbitol, acrylate polymers and copolymers, as well as pectin, dextrin and gelatin.

Other excipients include binders, such as acacia gum, pregelatinized starch, sodium alginate, glucose and other binders used in wet and dry granulation and direct compression tableting processes; disintegrants such as sodium starch glycolate, crospovidone, low-substituted hydroxypropyl cellulose and others; lubricants like magnesium and calcium stearate and sodium stearyl fumarate; flavorings; sweeteners; preservatives; pharmaceutically acceptable dyes and glidants such as silicon dioxide.

### High Performance Liquid Chromatography (HPLC):

### The Purity was Measured by High Performance Liquid Chromatography (HPLC) Under the Following Conditions:

Column: Unison C18 (150×4.6 mm)×3μ; Make: Imtak corporation P/N: UK005
Detector: UV at 220 nm
Flow rate: 0.80 mL/min
Injection volume: 20.0 μL
Run time: 50 min
Column temperature: 40° C.
Elution: Gradient

The following examples are given for the purpose of illustrating the present disclosure and should not be considered as limitation on the scope or spirit of the disclosure.

### EXAMPLES

### Example 1

### Preparation of pure (+)-N,N-Diisopropyl-3-(2-hydroxy-5-hydroxymethyl phenyl)-3-phenyl propylamine

### Step-I: Preparation of (+)-N,N-Diisopropyl-3-(2-benzyloxy-5-hydroxymethyl phenyl)-3-phenylpropylamine

(−)-N,N-Diisopropyl-3-(2-benzyloxy-5-carbomethoxyphenyl)-3-phenylpropylamine (200 g) was added to tetrahydrofuran (1000 ml), followed by the portion wise addition of lithium aluminium hydride (13.25 g) at below 10° C. over a period of 2 hours, followed by stirring for 1 hour at 10° C. This was followed by drop wise addition of ethyl acetate (400 ml) and water (200 ml) at below 5° C. The resulting mass was extracted with methylene chloride (1000 ml) and then stirred for 10 minutes. The organic layer was separated and then distilled to produce 189 g of (+)-N,N-Diisopropyl-3-(2-benzyloxy-5-hydroxymethylphenyl)-3-phenylpropylamine as an oily residue [HPLC Purity: 94.0%; Content of (+)-N,N-Diisopropyl-3-(2-benzyloxy-5-methylphenyl)-3-phenylpropylamine (dehydroxy impurity): 5.62%].

### Step-II: Preparation of crude (+)-N,N-Diisopropyl-3-(2-hydroxy-5-hydroxymethyl phenyl)-3-phenyl propylamine

Methanol (1000 ml) and (+)-N,N-diisopropyl-3-(2-benzyloxy-5-hydroxymethylphenyl)-3-phenylpropylamine (100 g, 232 mmole, obtained in step-I) were taken into a Parhydrogenator. Palladium carbon (5%, 20 g) was added and the mixture was hydrogenated under 2-3 kg pressure at 50-55° C. until completion of the reaction. The resulting mass was then filtered and the solvent was removed by vacuum at below 50° C. The resulting oil was dissolved in methylene chloride (100 ml) and the methylene chloride solution was washed with water, dried over sodium sulfate followed by evaporation to give crude (+)-N,N-Diisopropyl-3-(2-hydroxy-5-hydroxymethylphenyl)-3-phenylpropylamine as colorless oil. [Oil weight: 78 g; HPLC Purity: 98.5%; Content of (+)-N,N-Diisopropyl-3-(2-hydroxy-5-methylphenyl)-3-phenyl propylamine (dehydroxy impurity): 0.43%].

### Step-III: Purification of crude (+)-N,N-Diisopropyl-3-(2-hydroxy-5-hydroxymethyl phenyl)-3-phenyl propylamine:

Crude (+)-N,N-Diisopropyl-3-(2-hydroxy-5-hydroxymethylphenyl)-3-phenylpropylamine (78 g, obtained in step-II) was added to ethyl acetate (78 ml) at 25-30° C. under stirring followed by heating the mass at 50-55° C. to form a clear solution. The solution was cooled to 40° C. followed by the addition of petroleum ether (400 ml) over a period of 30 minutes. The resulting mass was cooled to 20-25° C. and stirred for 3 hours. The separated solid was filtered and then dried under vacuum at 40-45° C. to produce 70 gm of pure (+)-N,N-Diisopropyl-3-(2-hydroxy-5-hydroxymethylphenyl)-3-phenylpropylamine [HPLC Purity: 98.89%; Content of (+)-N,N-Diisopropyl-3-(2-hydroxy-5-methylphenyl)-3-phenyl propylamine (over reduced impurity or dehydroxy impurity): 0.1%].

### Example 2

### Preparation of 2-[(1R)-3-[bis(1-methylethyl)amino]-1-phenylpropyl]-4-hydroxy methylphenyl isobutyrate mandelate salt (Fesoterodine Mandelate)

(+)-N,N-Diisopropyl-3-(2-hydroxy-5-hydroxymethylphenyl)-3-phenylpropyl amine (100 g, 292 mmole, obtained in step-III of example 1) was added to methylene chloride (1200 ml) at 25-30° C. under stirring and the resulting mass was cooled to −10° C. This was followed by the drop wise addition of a solution of isobutyryl chloride (36 g, 292 mmole) dissolved in methylene chloride (800 ml) at −10 to −5° C. over a period of 1 hour. The resulting mass was stirred for 30 minutes at −10 to −5° C. and then raised the mass temperature to 0° C. followed by the addition of water (300 ml). The resulting mixture was stirred for 5 minutes and the organic layer was separated. This was followed by the addition of aqueous sodium bicarbonate solution (28.5 g of NaHCO₃ in 400 ml of water), stirred for 15 minutes and separated the organic layer followed by washing with water (300 ml). The organic layer was distilled under vacuum up to maximum extent and isopropyl alcohol (500 ml) was added to the residue. The temperature of the resulting mass was raised to 55° C. followed by the addition of L-(+)-mandelic acid (42.5 g) and stirred for 30 minutes at the same temperature. The resulting mass was cooled to 25-30° C. followed by stirred for 6 hours. The reaction mass was cooled further to 10-15° C. and stirred for 1 hour. The separated solid was filtered, washed the solid with chilled isopropyl alcohol (100 ml) and then dried the product under vacuum at 50° C. to produce 85 g of fesoterodine mandelate [HPLC Purity: 99.89%; Content of 2-[(1R)-3-[bis(1-methylethyl)amino]-1-phenylpropyl]-4-methylphenyl isobutyrate (dehydroxy impurity): Below Detection Limit].

### Example 3

### Preparation of 2-[(1R)-3-[bis(1-methylethyl)amino]-1-phenylpropyl]-4-hydroxy methylphenyl isobutyrate (Fesoterodine Free Base)

Fesoterodine mandelate (58 g, obtained in example 2) was dissolved in methylene chloride (290 ml) followed by the addition of 6% aqueous sodium bicarbonate (275 ml) and then stirred for 10 minutes. The separated methylene chloride layer was distilled under vacuum to get pure fesoterodine free base as oil (42 g).

### Example 4

### Preparation of Fesoterodine Fumarate

A solution of fesoterodine free base (42 g, obtained in example 3) in methyl ethyl ketone (90 ml) was stirred with fumaric acid (12 g) at 80° C. for 1 hour. Cyclohexane (30 ml) was slowly added to the mass under stirring and the stirring was continued for one hour at 80° C. The resulting mass was cooled slowly to 25-30° C. and stirred for 12 hours at the same temperature. The solution was further cooled to 0-5° C. and then stirred for 12 hours. The solid separated was filtered and washed with the mixture of cyclohexane and methyl ethyl ketone to produce 39 g of fesoterodine fumarate [HPLC Purity: 99.91%; Content of 2-[(1R)-3-[bis(1-methylethyl)amino]-1-phenylpropyl]-4-methylphenyl isobutyrate (dehydroxy impurity): Below Detection Limit].

### Example 5

### Preparation of 2-[(1R)-3-[bis(1-methylethypamino]-1-phenylpropyl]-4-methyl phenyl isobutyrate (Fesoterodine Dehydroxy Impurity or Dehydroxyfesoterodine)

Methylene chloride (100 ml) was added to (+)-N,N-Diisopropyl-3-(2-hydroxy-5-methylphenyl)-3-phenylpropylamine (8 g) at 25-30° C. and then the resulting mass was cooled to −10° C. This was followed by drop wise addition of a solution of isobutyryl chloride (3.1 g) dissolved in methylene chloride (65 ml) at −10 to −5° C. over a period of 1 hour. The resulting mass was stirred for 30 minutes at −10 to −5° C. and then raised the mass temperature to 0° C. followed by the addition of water (300 ml). The resulting mass was stirred for 5 minutes followed by separation of the organic layer. 5% Aqueous sodium bicarbonate solution was added to the resulting organic layer and stirred for 15 minutes. The resulting organic layer was separated followed by washing with water (300 ml). The organic layer was distilled under vacuum up to maximum extent to give 2-[(1R)-3-[bis(1-methylethyl)amino]-1-phenylpropyl]-4-methyl phenyl isobutyrate as oily residue. (Oil weight. 9.0 g; HPLC Purity: 97.58%].

### Example 6

### Purification of Crude (+)-N,N-Diisopropyl-3-(2-hydroxy-5-hydroxymethylphenyl)-3-phenyl-propylamine

Ethyl acetate (140 ml) was added to crude (+)-N,N-Diisopropyl-3-(2-hydroxy-5-hydroxy methylphenyl)-3-phenylpropylamine (140 g) at 25-30° C. under stirring and the resulting mixture was heated to 50-55° C. to form a clear solution. The solution was cooled to 20-25° C. followed by slow addition of n-hexane (700 ml) over a period of 30 minutes and then stirred for 3 hours at 20-25° C. The solid precipitated was filtered, washed with the mixture of ethyl acetate (56 ml) and n-hexane (224 ml) and then dried the product in air oven to produce 112 g of pure (+)-N,N-Diisopropyl-3-(2-hydroxy-5-hydroxy methyl phenyl)-3-phenylpropylamine [HPLC Purity: 99.2%; Content of (+)-N,N-Diisopropyl-3-(2-hydroxy-5-methylphenyl)-3-phenyl propylamine (dehydroxy impurity): 0.1%].

### Example 7

### Preparation of Fesoterodine Fumarate

Fesoterodine fumarate (10 g, obtained in example 4) was suspended in isopropyl alcohol (40 ml) followed by heating at reflux to provide a clear solution. The solution was cooled to 40° C. over a period of one hour followed by drop wise addition of isopropyl ether (80 ml) at 38-40° C. The resulting mass was stirred for 15 hours at 38-40° C. The separated solid was filtered under nitrogen atmosphere, washed with isopropyl ether (20 ml) and then dried the material under vacuum at 40-45° C. for 12 hours to produce 6 g of pure fesoterodine fumarate with desired particle size [Purity by HPLC: 99.8%; Particle Size Data: (D₁₀): 4.243 μm, (D₅₀): 14.875 μm, and (D₉₀): 90.272 μm].

### Example 8

### Preparation of Fesoterodine Fumarate

Fesoterodine (15 g) was dissolved in isopropyl alcohol (60 ml) followed by the addition of fumaric acid (4.0 g). The resulting mass was heated to 55-60° C. and then stirred to form a clear solution. The reaction mass was cooled to 25-30° C. over a period of one hour followed by the addition of isopropyl ether (180 ml) and then stirred for 17 hours at 25-30° C. The separated solid was filtered under nitrogen atmosphere, washed with isopropyl ether (60 ml) and then dried the material under vacuum at 40-45° C. for 12 hours to produce 15 g of pure fesoterodine fumarate [Purity by HPLC: 99.85%; Particle Size Data: (D₁₀): 4.518 μm, (D₅₀): 22.291μm (D₉₀): 85.415 μm].

### Example 9

### Preparation of Fesoterodine Fumarate

Fesoterodine (15 g) was added to isopropyl alcohol (60 ml) and then heated to 55-60° C. followed by the addition of fumaric acid (4.0 g). The resulting solution was stirred at 55-60° C. for one hour and then cooled to 25-30° C. over a period of one hour. Isopropyl ether (180 ml) was slowly added to the resulting mass over a period of one hour and stirred for 6 hours at 25-30° C. for complete crystallization. The separated solid was filtered under nitrogen atmosphere, washed with isopropyl ether (60 ml) and then dried the material under vacuum at 40-45° C. for 12 hours to produce 15 g of pure fesoterodine fumarate [Purity by HPLC: 99.85%; Particle Size Data: (D₁₀): 4.485 μm, (D₅₀): 31.876 μm, and (D₉₀): 110.282 μm].

### Example 10

Fesoterodine fumarate (obtained from any one of examples 7-9) was fine-milled by being passed through a grinder (Make: Morphy Richards, Model-Icon DLX) having stainless steel liquidizing blade for 3-4 minutes to obtain 90 volume-% of the fesoterodine fumarate particles having a diameter of less than about 50 microns.

Unless otherwise indicated, the following definitions are set forth to illustrate and define the meaning and scope of the various terms used to describe the invention herein.

The term “pharmaceutically acceptable” means that which is useful in preparing a pharmaceutical composition that is generally non-toxic and is not biologically undesirable and includes that which is acceptable for veterinary use and/or human pharmaceutical use.

The term “pharmaceutical composition” is intended to encompass a drug product including the active ingredient(s), pharmaceutically acceptable excipients that make up the carrier, as well as any product which results, directly or indirectly, from combination, complexation or aggregation of any two or more of the ingredients. Accordingly, the pharmaceutical compositions encompass any composition made by admixing the active ingredient, active ingredient dispersion or composite, additional active ingredient(s), and pharmaceutically acceptable excipients.

The term “therapeutically effective amount” as used herein means the amount of a compound that, when administered to a mammal for treating a state, disorder or condition, is sufficient to effect such treatment. The “therapeutically effective amount” will vary depending on the compound, the disease and its severity and the age, weight, physical condition and responsiveness of the mammal to be treated.

The term “delivering” as used herein means providing a therapeutically effective amount of an active ingredient to a particular location within a host causing a therapeutically effective blood concentration of the active ingredient at the particular location. This can be accomplished, e.g., by topical, local or by systemic administration of the active ingredient to the host.

The term “buffering agent” as used herein is intended to mean a compound used to resist a change in pH upon dilution or addition of acid of alkali. Such compounds include, by way of example and without limitation, potassium metaphosphate, potassium phosphate, monobasic sodium acetate and sodium citrate anhydrous and dehydrate and other such material known to those of ordinary skill in the art.

The term “sweetening agent” as used herein is intended to mean a compound used to impart sweetness to a formulation. Such compounds include, by way of example and without limitation, aspartame, dextrose, glycerin, mannitol, saccharin sodium, sorbitol, sucrose, fructose and other such materials known to those of ordinary skill in the art.

The term “binders” as used herein is intended to mean substances used to cause adhesion of powder particles in granulations. Such compounds include, by way of example and without limitation, acacia, alginic acid, tragacanth, carboxymethylcellulose sodium, polyvinylpyrrolidone, compressible sugar (e.g., NuTab), ethylcellulose, gelatin, liquid glucose, methylcellulose, pregelatinized starch, starch, polyethylene glycol, guar gum, polysaccharide, bentonites, sugars, invert sugars, poloxamers (PLURONIC™ F68, PLURONIC™ F127), collagen, albumin, celluloses in non-aqueous solvents, polypropylene glycol, polyoxyethylene-polypropylene copolymer, polyethylene ester, polyethylene sorbitan ester, polyethylene oxide, microcrystalline cellulose, combinations thereof and other material known to those of ordinary skill in the art.

The term “diluent” or “filler” as used herein is intended to mean inert substances used as fillers to create the desired bulk, flow properties, and compression characteristics in the preparation of solid dosage formulations. Such compounds include, by way of example and without limitation, dibasic calcium phosphate, kaolin, sucrose, mannitol, microcrystalline cellulose, powdered cellulose, precipitated calcium carbonate, sorbitol, starch, combinations thereof and other such materials known to those of ordinary skill in the art.

The term “glidant” as used herein is intended to mean agents used in solid dosage formulations to improve flow-properties during tablet compression and to produce an anti-caking effect. Such compounds include, by way of example and without limitation, colloidal silica, calcium silicate, magnesium silicate, silicon hydrogel, cornstarch, talc, combinations thereof and other such materials known to those of ordinary skill in the art.

The term “lubricant” as used herein is intended to mean substances used in solid dosage formulations to reduce friction during compression of the solid dosage. Such compounds include, by way of example and without limitation, calcium stearate, magnesium stearate, mineral oil, stearic acid, zinc stearate, combinations thereof and other such materials known to those of ordinary skill in the art.

The term “disintegrant” as used herein is intended to mean a compound used in solid dosage formulations to promote the disruption of the solid mass into smaller particles which are more readily dispersed or dissolved. Exemplary disintegrants include, by way of example and without limitation, starches such as corn starch, potato starch, pregelatinized, sweeteners, clays, such as bentonite, microcrystalline cellulose (e.g., Avicel™), carsium (e.g., Amberlite™), alginates, sodium starch glycolate, gums such as agar, guar, locust bean, karaya, pectin, tragacanth, combinations thereof and other such materials known to those of ordinary skill in the art.

The term “wetting agent” as used herein is intended to mean a compound used to aid in attaining intimate contact between solid particles and liquids. Exemplary wetting agents include, by way of example and without limitation, gelatin, casein, lecithin (phosphatides), gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters, (e.g., TWEEN™s), polyethylene glycols, polyoxyethylene stearates colloidal silicon dioxide, phosphates, sodium dodecylsulfate, carboxymethylcellulose calcium, carboxymethylcellulose sodium, methylcellulose, hydroxyethylcellulose, hydroxyl propylcellulose, hydroxypropylmethylcellulose phthalate, noncrystalline cellulose, magnesium aluminum silicate, triethanolamine, polyvinyl alcohol, and polyvinylpyrrolidone (PVP). Tyloxapol (a nonionic liquid polymer of the alkyl aryl polyether alcohol type) is another useful wetting agent, combinations thereof and other such materials known to those of ordinary skill in the art.

The term “micronization” used herein means a process or method by which the size of a population of particles is reduced.

As used herein, the term “micron” or “μm” both are same refers to “micrometer” which is 1×10⁻⁶ meter.

As used herein, “crystalline particles” means any combination of single crystals, aggregates and agglomerates.

As used herein, “Particle Size Distribution (PSD)” means the cumulative volume size distribution of equivalent spherical diameters as determined by laser diffraction in Malvern Master Sizer 2000 equipment or its equivalent. “Mean particle size distribution, i.e., (D₅₀)” correspondingly, means the median of said particle size distribution.

The important characteristics of the PSD were the (D₉₀), which is the size, in microns, below which 90% of the particles by volume are found, and the (D₅₀), which is the size, in microns, below which 50% of the particles by volume are found.

As used herein, “blend uniformity” refers to the homogeneity of granulate including fesoterodine fumarate particles before tablet formulation, and can represent one sample or the average of more than one sample.

The term “crude fesoterodine or a pharmaceutically acceptable salt thereof” as used herein refers to fesoterodine or a pharmaceutically acceptable salt thereof containing greater than about 0.2 area-%, more specifically greater than about 0.3 area-%, still more specifically greater than about 1 area-% and most specifically greater than about 3 area-% of the dehydroxy impurity of I(i).

As used herein, the term, “detectable” refers to a measurable quantity measured using an HPLC method having a detection limit of 0.01 area-%.

As used herein, in connection with amount of impurities in fesoterodine or a pharmaceutically acceptable salt thereof, the term “not detectable” means not detected by the herein described HPLC method having a detection limit for impurities of 0.01 area-%.

As used herein, “limit of detection (LOD)” refers to the lowest concentration of analyte that can be clearly detected above the base line signal, is estimated is three times the signal to noise ratio.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1.-3. (canceled)

4. Fesoterodine or a pharmaceutically acceptable salt thereof comprising a fesoterodine dehydroxy impurity, 2-[(1R)-3-[bis(1-methylethyl)amino]-1-phenylpropyl]-4-methyl phenyl isobutyrate, of formula I(i): 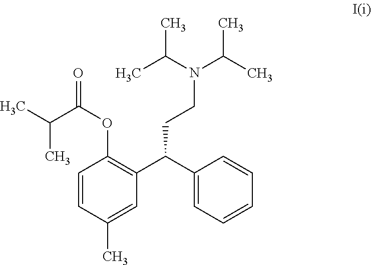
in an amount of less than about 0.2 area-% as measured by HPLC.

5. (canceled)

6. Fesoterodine of claim 4, comprising the fesoterodine dehydroxy impurity in an amount of about 0.01 area-% to about 0.15 area-%; wherein the fesoterodine or a pharmaceutically acceptable salt thereof has a total purity of about 98% to about 99.99% as measured by HPLC; and wherein the pharmaceutically acceptable salt of fesoterodine is selected from the group consisting of hydrochloride, hydrobromide, nitrate, sulfate, mandelate, oxalate, succinate, maleate, besylate, tosylate, palmitate, fumarate and tartarate.

7. Fesoterodine of claim 4, having a non-detectable amount of fesoterodine dehydroxy impurity as measured by HPLC.

8. (canceled)

9. (canceled)

10. A process for preparing highly pure fesoterodine or a pharmaceutically acceptable salt thereof having less than 0.2 area-% of fesoterodine dehydroxy impurity of claim 4, comprising:
a) providing a solution of crude (+)-N,N-diisopropyl-3-(2-hydroxy-5-hydroxymethyl phenyl)-3-phenylpropylamine in a first solvent, wherein the first solvent is selected from the group consisting of water, an alcohol, an ester, acetone, acetonitrile, N,N-dimethylformamide, N,N-dimethylacetamide, dimethylsulfoxide, and mixtures thereof;
b) optionally, subjecting the solution obtained in step-(a) to carbon treatment or silica gel treatment;
c) admixing the solution with an anti-solvent to form a precipitate, wherein the anti-solvent is selected from the group consisting of an ether, a hydrocarbon, and mixtures thereof;
d) recovering pure (+)-N,N-diisopropyl-3-(2-hydroxy-5-hydroxymethylphenyl)-3-phenylpropylamine containing less than 0.2 area-% of dehydroxy impurity, (+)-N,N-diisopropyl-3-(2-hydroxy-5-methylphenyl)-3-phenylpropylamine from the precipitate;
e) condensing the pure compound of formula II obtained in step-(d) with isobutyryl chloride in a second solvent, optionally in the presence of a base, to produce pure fesoterodine containing less than 0.2 area-% of fesoterodine dehydroxy impurity, wherein the second solvent is selected from the group consisting of a hydrocarbon, a chlorinated hydrocarbon, a nitrile, an ester, an ether, and mixtures thereof; and
f) optionally, converting the pure fesoterodine base obtained in step-(e) into a pharmaceutically acceptable acid addition salt thereof; or
g) optionally, converting the pure fesoterodine base obtained in step-(e) into its mandelate salt and then converting the fesoterodine mandelate salt formed into a pharmaceutically acceptable acid addition salt of fesoterodine.

11. (canceled)

12. The process of claim 10, wherein the first solvent used in step-(a) is selected from the group consisting of water, methanol, ethanol, n-propanol, isopropyl alcohol, isobutanol, n-butanol, tert-butanol, amyl alcohol, isoamyl alcohol, hexanol, ethyl acetate, isopropyl acetate, n-butyl acetate, tert-butyl acetate, acetone, acetonitrile, N,N-dimethylformamide, N,N-dimethylacetamide, dimethylsulfoxide, and mixtures thereof; wherein the anti-solvent used in step-(c) is selected from the group consisting of tetrahydrofuran, dioxane, diethyl ether, diisopropyl ether, monoglyme, diglyme, n-pentane, n-hexane, n-heptane and their isomers, petroleum ether, cyclohexane, toluene, xylene, and mixtures thereof; wherein the second solvent used in step-(e) is methylene chloride; and wherein the base used in step-(e) is an organic or inorganic base.

13. The process of claim 12, wherein the first solvent is selected from the group consisting of ethyl acetate, isopropyl alcohol, and mixtures thereof; wherein the anti-solvent is selected from the group consisting of diisopropyl ether, n-hexane, petroleum ether and mixtures thereof; and wherein the base used in step-(e) is selected from the group consisting of triethylamine, sodium hydroxide, calcium hydroxide, magnesium hydroxide, potassium hydroxide, lithium hydroxide, sodium carbonate, potassium carbonate, lithium carbonate, sodium tert-butoxide, sodium isopropoxide and potassium tert-butoxide.

14. The process of claim 10, wherein the solution in step-(a) is provided by dissolving crude (+)-N,N-diisopropyl-3-(2-hydroxy-5-hydroxymethyl phenyl)-3-phenylpropyl amine in the first solvent at a temperature of about 25° C. to the reflux temperature of the solvent used; wherein the carbon treatment or silica gel treatment in step-(b) is carried out by stirring the solution with finely powdered carbon or silica gel at a temperature of below about 70° C. for at least 15 minutes, and filtering the resulting mixture through a filtration bed to obtain a filtrate containing the compound by removing charcoal; wherein recovering in step-(d) is carried out by filtration, filtration under vacuum, decantation, centrifugation, filtration employing a filtration media of a silica gel or celite, or a combination thereof; wherein the condensation reaction in step-(e) is carried out at a temperature of about −20° C. to about 30° C. for at least 20 minutes; wherein the conversion of fesoterodine base to fesoterodine mandelate in step-(g) is carried out by providing a solution of fesoterodine free base in an alcoholic solvent, adding mandelic acid to the solution and then isolating pure fesoterodine mandelate having less than 0.2 area-% of a fesoterodine dehydroxy impurity; and wherein the pure fesoterodine mandelate obtained is further converted to a pharmaceutically acceptable acid addition salt of fesoterodine by treating the fesoterodine mandelate with a base in an organic solvent to liberate fesoterodine free base and then converting the fesoterodine free base into its pharmaceutically acceptable salts thereof.

15. (canceled)

16. The process of claim 10, wherein the solution in step-(a) is prepared by a process comprising:
a) reducing (−)-N,N-diisopropyl-3-(2-benzyloxy-5-carbomethoxyphenyl)-3-phenyl propylamine with lithium aluminium hydride, optionally in the presence of an organic or inorganic base, in a solvent to produce crude (+)-N,N-diisopropyl-3-(2-benzyloxy-5-hydroxymethylphenyl)-3-phenylpropylamine;
b) hydrogenating the crude (+)-N,N-diisopropyl-3-(2-benzyloxy-5-hydroxymethyl phenyl)-3-phenylpropylamine in the presence of a hydrogenation catalyst in a solvent to produce a reaction mass containing crude (+)-N,N-diisopropyl-3-(2-hydroxy-5-hydroxymethylphenyl)-3-phenylpropylamine;
c) subjecting the reaction mass obtained in step-(b) to washings, extractions, evaporations or a combination thereof to isolate crude (+)-N,N-diisopropyl-3-(2-hydroxy-5-hydroxymethylphenyl)-3-phenylpropylamine; and
d) dissolving or extracting the crude (+)-N,N-diisopropyl-3-(2-hydroxy-5-hydroxymethylphenyl)-3-phenylpropylamine obtained in step-(c) in the first solvent at a temperature of about 25° C. to the reflux temperature of the solvent used.

17.-37. (canceled)

38. A process for preparing highly pure fesoterodine or a pharmaceutically acceptable salt thereof having less than 0.2 area-% of fesoterodine dehydroxy impurity of claim 4, comprising:
a) providing a solution of crude fesoterodine or its mandelate salt in a solvent selected from the group consisting of water, an alcohol, an ester, acetone, acetonitrile, dimethylsulfoxide, dimethylformamide, dimethylacetamide, and mixtures thereof;
b) optionally, subjecting the solution obtained in step-(a) to carbon treatment or silica gel treatment;
c) admixing the solution with an anti-solvent to produce a reaction mass; and
d) recovering pure fesoterodine or its mandelate salt having less than 0.2 area-% of fesoterodine dehydroxy impurity from the reaction mass, and optionally converting the pure fesoterodine or its mandelate formed into a pharmaceutically acceptable acid addition salt thereof.

39.-43. (canceled)

44. A process for preparing dehydroxyfesoterodine impurity, 2-[(1R)-3-[bis(1-methylethyl)-amino]-1-phenylpropyl]-4-methylphenyl isobutyrate, of formula I(i) as defined in claim 4, 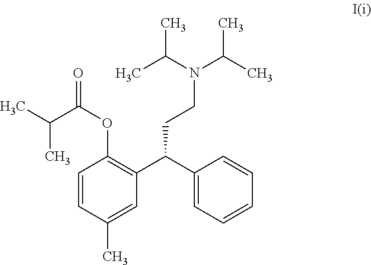
comprising condensing N,N-diisopropyl-3-(2-hydroxy-5-methylphenyl)-3-phenyl propylamine (tolterodine) of formula II(i): 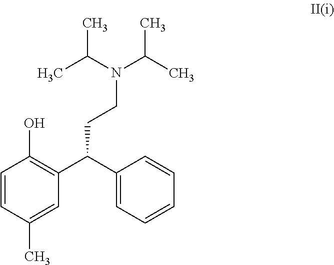
with isobutyryl chloride, optionally in the presence of a base, in a solvent to provide the 2-[(1R)-3-[bis(1-methylethyl)amino]-1-phenylpropyl]-4-methylphenyl isobutyrate of formula I(i), wherein the solvent is selected from the group consisting of an alcohol, a ketone, a cyclic ether, an aliphatic ether, a hydrocarbon, a chlorinated hydrocarbon, a nitrile, an ester, and mixtures thereof, and wherein the base is an organic or inorganic base.

45. (canceled)

46. The process of claim 44, wherein the solvent is selected from the group consisting of n-hexane, n-heptane, cyclohexane, toluene, xylene, methylene chloride, ethyl dichloride, chloroform, and mixtures thereof; wherein the condensation reaction is carried out at a temperature of about −20° C. to about 30° C. for at least 20 minutes; and wherein the compound of formula I(i) obtained is isolated by substantially complete evaporation of the solvent, concentrating the solution or distillation of solvent under inert atmosphere, cooling, partial removal of the solvent from the solution, addition of a precipitating solvent, or a combination thereof.

47.-53. (canceled)

54. Fesoterodine fumarate has a D₉₀ particle size of less than or equal to about 200 microns.

55. Fesoterodine fumarate of claim 54, having a D₉₀ particle size of about 1 micron to about 190 microns; and further comprising the fesoterodine dehydroxy impurity in an amount of less than about 0.2 area-% as measured by HPLC.

56. (canceled)

57. (canceled)

58. A process for the preparation of fesoterodine fumarate having a D₉₀ particle size of less than or equal to about 200 microns of claim 54, comprising:
providing a solution of fesoterodine fumarate in an alcohol solvent, wherein the alcohol solvent is selected from the group consisting of methanol, ethanol, n-propanol, isopropyl alcohol, n-butanol, tert-butanol, amyl alcohol, hexanol, and mixtures thereof;
optionally, filtering the solvent solution to remove any extraneous matter;
optionally, seeding the solution;
admixing the solution with an anti-solvent to produce a reaction mass, wherein the anti-solvent is an ether solvent selected from the group consisting of diisopropyl ether, diethyl ether, tetrahydrofuran, dioxane, monoglyme, diglyme, and mixtures thereof; and
recovering fesoterodine fumarate particles substantially free of dehydroxy impurity having a D₉₀ particle size of about 80 microns to about 200 microns from the reaction mass obtained in step-(d).

59. (canceled)

60. The process of claim 58, wherein the alcohol solvent used in step-(a) is isopropyl alcohol; wherein the anti-solvent is diisopropyl ether; and wherein the solution obtained in step-(a) is optionally subjected to carbon treatment or silica gel treatment.

61.-73. (canceled)

74. The process of claim 58, further comprising a process for controlling the particle size of fesoterodine fumarate, comprising:
providing solid particles of fesoterodine fumarate having a D₉₀ particle size of about 80 microns to about 200 microns; and
milling the fesoterodine fumarate of step-(a) to obtain fesoterodine fumarate having a D₉₀ particle size of about 1 micron to about 190 microns.

75. (canceled)

76. A pharmaceutical composition comprising highly pure fesoterodine or a pharmaceutically acceptable salt thereof having a fesoterodine dehydroxy impurity in an amount of less than about 0.2 area-% of claim 4 and one or more pharmaceutically acceptable excipients.

77. (canceled)

78. The pharmaceutical composition of claim 76, wherein the fesoterodine or a pharmaceutically acceptable salt thereof contains a non-detectable amount of fesoterodine dehydroxy impurity as measured by HPLC.

79.-82. (canceled)

83. A pharmaceutical composition comprising fesoterodine fumarate has a D₉₀ particle size of less than or equal to about 200 microns of claim 54, and one or more pharmaceutically acceptable excipients.

84. The pharmaceutical composition of claim 83, wherein the D₉₀ particle size is about 5 microns to about 150 microns, and wherein the fesoterodine fumarate contains the fesoterodine dehydroxy impurity in an amount of less than about 0.2 area-% as measured by HPLC.

85.-88. (canceled)
